(19) United States
(12) Patent Application Publication
(10) Pub. No.: US 2014/0140969 A1
(43) Pub. Date: May 22, 2014

(54) METHODS AND COMPOSITIONS FOR MUSCULAR DYSTROPHIES

(71) Applicants: Sangamo BioSciences, Inc., Richmond, CA (US); Universite De Montreal, Montreal (CA)

(72) Inventors: Christian Beausejour, Montreal (CA); Michael C. Holmes, Richmond, CA (US)

(21) Appl. No.: 14/085,558

(22) Filed: Nov. 20, 2013

Related U.S. Application Data
(60) Provisional application No. 61/728,735, filed on Nov. 20, 2012.

(51) Int. Cl. A61K 35/34; C12N 15/85

(52) U.S. Cl. CPC A61K 35/34; C12N 15/85
USPC 424/93.21; 435/325

(57) ABSTRACT

Methods and compositions for editing of genes involved in muscular dystrophies.

### CROSS-REFERENCE TO RELATED APPLICATIONS

The present application claims the benefit of U.S. Provisional Application No. 61/728,735, filed Nov. 20, 2012, the disclosure of which is hereby incorporated by reference in its entirety.

### TECHNICAL FIELD

The present disclosure is in the fields of genome editing and therapeutics.

### BACKGROUND

Engineered nucleases, including zinc finger nucleases, TALENs and homing endonucleases designed to specifically bind to target DNA sites are useful in genome engineering. For example, zinc finger nucleases (ZFNs) are proteins comprising engineered site-specific zinc fingers fused to a nuclease domain and TALE-nucleases (TALENs) are proteins comprising engineered site-specific TAL-effector domains fused to a nuclease domain. Such ZFNs, TALENs have been successfully used for genome modification in a variety of different species. See, for example, U.S. Pat. No. 8,586,526; U.S. Patent Publications 20030232410; 20050208489; 20050026157; 20050064474; 20060188987; 20060063231; and International Publication WO 07/014275, the disclosures of which are incorporated by reference in their entireties for all purposes. These engineered nucleases cleave a target nucleotide sequence, which increases the frequency of homologous recombination at the targeted locus by more than 1000-fold. In addition, the repair of a site-specific DSB by non-homologous end joining (NHEJ) can also result in gene modification, including gene insertion. Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage.

Muscular dystrophies are diseases that are characterized by a progressive degeneration and weakening of muscle groups. One well known muscular dystrophy is Duchenne's muscular dystrophy, which is an X-linked disease that afflicts 1 in every 3500 boys. It is caused by the lack of the protein dystrophin in the individual muscle cells, and symptoms first appear when the child is approximately 3 years old, and depending on the severity of the disease, death can occur when the patient is in his twenties. The gene encoding dystrophin, DMD, is extremely large and covers 2.4 megabases of DNA comprising 79 exons that encode a 14 kb mRNA. The protein is an integral part of the dystophin-associated glycoprotein complex (DGC) which comprises the dystroglycans, sarcoglycans, integrins and caveolin. It appears that dystrophin's main function is to stabilize the DGC and link the muscle fiber cytoskeleton to the cell membrane. Mutant cells that do not produce dystrophin lack functioning DGCs (Pichavant et al (2011) Mol Ther 19(5): 830-840), and non-functional DGCs result in diminished levels of the DGC member proteins. This in turn leads to progressive fiber damage and membrane leakage. DMD patients are usually wheelchair bound by 12 years of age and die of respiratory failure in their late teens or early twenties. Additionally, mutations that occur in some DGC member proteins can also cause autosomally inherited forms of muscular dystrophy (Nowak and Davies (2004) EMBO Reports 5(9): 872-876).

In patients that lack functional dystrophin, approximately 40% have point mutations that cause a frameshift in the coding sequence such that during translation, a premature stop is encountered resulting in the production of a truncated or non-functioning protein. The other 60% have large insertions or deletions that also result in alteration of frame and similarly result in production of a non-functional protein (Nowak and Davies ibid). Patients with non-functional dystrophin have the most severe disease; which is also known as Duchenne's Muscular Dystrophy (DMD). Other patients, whose mutations are characterized by gene deletions of regions that encode internal portions of dystrophin, resulting in less functional dystrophin protein as compared to wild type, may have less severe disease, which is called Becker Muscular Dystrophy (BMD). BMD patients have been known to live into their 50's.

Currently there is no cure for any muscular dystrophy patient. It has been shown that corticosteroids provide some temporary benefit for DMD patients and may prolong walking a few years longer as compared to patients that do not take them (see Fairclough et al (2011) Exp Physiol 96 (11): 1101-1113). While helpful, these compounds provide only a temporary benefit for patients and have many unacceptable side effects when taken for long periods.

Adeno-associated virus-mediated in vivo gene transfer of the microdystrophin gene, a truncated but functional form of the protein, was recently shown to be feasible in dystrophic dogs (Wang et al (2012) Mol Ther 20: 1501-1507). Although promising, this strategy requires the in vivo administration of virus and immunosuppression of the host, procedures that can lead to multiple adverse effects.

Thus, there remains a need for treatments for muscular dystrophy, for example nucleases that can be used to introduce a dystrophin gene into a cell that can be used in research and therapeutic applications.

### SUMMARY

The present disclosure relates to development of reagents to introduce a gene into a cell for the treatment or prevention of a muscular dystrophy. In preferred embodiments, the cell is a stem cell or induced pluripotent stem cell (iPSC) which can then be differentiated into a myoblast and introduced into a muscle. In some embodiments, the stem cell is a muscle-derived stem cell (see Usas et al, (2011) Medicina (Kaunas) 47(9): 469-479). Nucleases, for example engineered meganucleases, zinc finger nucleases (ZFNs), TALE-nucleases (TALENs) and/or CRISPR/Cas nuclease systems are used to cleave DNA at a ‘safe harbor’ gene locus (e.g. CCR5, AAVS1, HPRT, Rosa or albumin) in the cell into which the gene is inserted. Targeted insertion of a donor transgene may be via homology directed repair (HDR) or non-homology repair mechanisms (e.g., NHEJ donor capture). The nuclease can induce a double-stranded (DSB) or single-stranded break (nick) in the target DNA. In some embodiments, two nickases are used to create a DSB by introducing two nicks. In some cases, the nickase is a ZFN, while in others, the nickase is a TALEN or a CRISPR/Cas system.

For the treatment or prevention of X-linked dystrophies (Duchenne's and Becker's), the inserted gene is a dystophin gene or a fragment and/or truncation thereof. For the treatment or prevention of autosomally inherited muscular dystrophies caused by a non-functional DCG member protein, the inserted gene encodes the functional DCG member protein. The present disclosure provides nucleases (e.g., ZFNs, TALENs and/or CRISPR/Cas systems) specific for safe harbor genes (e.g. CCR5, HPRT, AAVS1, Rosa or albumin, See, e.g., U.S. Pat. Nos. 7,951,925 and 8,110,379; U.S. Publication Nos. 20080159996; 201000218264; 20120017290; 20110265198; 20130137104; 20130122591; 20130177983 and 20130177960 and U.S. Provisional Application No. 61/823,689). In some embodiments, the safe harbor is the CCR5 gene.

In one aspect, the methods and compositions of the invention comprise engineered stem cells. In certain embodiments, the stem cells comprise a safe harbor specific nuclease (e.g., CCR5), while in further embodiments, the stem cells comprise a safe harbor specific nuclease and at least one transgene donor. In certain embodiments, the transgene donor encodes a dystrophin gene or fragment and/or truncation thereof. In some embodiments, the dystrophin gene is a mini-dystrophin gene, while in others the dystrophin gene is a micro-dystrophin gene (Pichavant (2011) ibid). In further embodiments, the dystrophin gene is a fragment and/or truncation that is not a mini-dystrophin or a micro-dystrophin, but is functional nonetheless. In some embodiments, the gene for insertion encodes a member of the DGC. In some embodiments, the transgene also comprises a transcriptional regulator while in others, it does not and transcription is regulated by an endogenous regulator. In another aspect, the methods of the invention comprise a composition for therapeutic treatment of a subject in need thereof. In some embodiments, the composition comprises engineered stem cells comprising a safe harbor specific nuclease, and a transgene donor encoding a dystrophin or fragment and/or truncation thereof

In another aspect, provided herein are methods and compositions for introducing a transcription factor or nuclease (e.g., zinc finger protein transcription factor, TAL-effector domain transcription factor, ZFN, TALEN or CRISPR/Cas nuclease) that is engineered to bind to a target site at a safe harbor locus (or polynucleotide encoding the fusion protein) into cells from a subject with a disease or disorder to prevent or treat a disease or disorder. In some embodiments, the disease or disorder is a muscular dystrophy. Non-limiting examples of muscular dystrophies that can be treated and/or prevented include Duchenne's muscular dystrophy and Becker's muscular dystrophy.

### BRIEF DESCRIPTION OF THE DRAWINGS

FIG. 1, panels A through D, show targeted addition of the eGFP gene in human myoblasts. FIG. 1A is a schematic representation of (1) Ad5/F35 chimeric adenoviral vector containing the expression cassette for the CCR5 specific ZFN, (2) the location of the ZFN target site in exon 3 of the endogenous CCR5 locus, (3) IDLV donor vector containing the PGK-eGFP expression cassette flanked by CCR5-homology arms, (4) the expected result after targeted gene addition at the CCR5 locus. PCR primers (black arrows) used for targeted integration analysis are indicated. FIG. 1B shows levels of targeted-addition obtained in human myoblasts as determined by flow cytometry on day 3 post-transduction with increasing doses of Ad5/F35 ZFN and IDLV.GFP donor vector. Shown is the average and standard deviation of three independent experiments. FIG. 1C shows long term profile of eGFP gene expression in myoblasts transduced with the optimal dose of IDLV donor DNA and AdZFN vectors. Note the dilution over time of the eGFP expression in absence of AdZFN. FIG. 1D are representative photographs showing the expression of eGFP in myoblasts four weeks following their transduction with the indicated vectors. Nuclei were stained with DAPI. Results are representative of three independent experiments. Magnification: 100×.

FIG. 2, panels A through C, show targeted addition of the microdystrophin gene in human myoblasts. FIG. 2A depicts schematics of IDLV donor DNA vector containing the PGK-microdystrophin-V5 expression cassette flanked by CCR5-homology arms (top) and of the expected result after targeted gene addition at the CCR5 locus of the microdystrophin-V5 donor DNA. PCR primers (black arrows) used for targeted integration analysis are indicated (bottom). FIG. 2B shows myoblasts that were transduced with the indicated viral vectors and the expression of the microdystrophin-V5 was detected by immuno-fluorescence using a V5 specific antibody (in red). Nuclei were counterstained with DAPI. Myoblasts were immuno-stained four weeks post-transduction to allow for the dilution of the IDLV vector. The magnification is 200×. FIG. 2C is a quantitative representation of the levels of microdystrophin-V5 gene targeted myoblasts four weeks post-transduction. The proportion of V5-positive cells was determined by counting manually a total of 300 treated cells in randomly selected fields. Results are representative of three independent experiments. “NTC” refers to non-transduced cells.

FIG. 3, panels A and B, show targeted integration of the eGFP and the microdystrophin genes at the CCR5 locus. FIG. 3A shows myoblasts that were transduced with the indicated vectors and expanded in vitro for four weeks before genomic DNA was collected. Targeted integration (TI) was determined by PCR using a set of primers specific for the 5′ integration junction (as shown in FIG. 1A and 2A). The bottom gel shows the amplification of the GAPDH gene used as an internal control. NTC: non-transduced cells. FIG. 3B shows myoblasts that were transduced as described above using the indicated vector concentrations and evidence for ZFN-targeted gene disruption at the CCR5, CCR2 and ABLIM2 loci determined by the Cel-1 Surveyor assay.

FIG. 4, panels A through E, show selective in vitro enrichment of gene targeted myoblasts using the MGMT^{P140K} drug resistance gene. FIG. 4A shows schematics of IDLV donor DNA vector containing the PGK-MGMT^{P140K}-2A-GFP expression cassette flanked by CCR5-homology arms (top) and of the expected result after targeted gene addition at the CCR5 locus of the MGMT^{P140K}-2A-GFP donor DNA (bottom). FIG. 4B is a histogram showing the levels of targeted MGMT^{P140K}-2A-GFP gene expression obtained in human myoblasts transduced with the indicated doses of vector as determined by flow cytometry four weeks post-transduction. Shown is the average and standard deviation of three independent experiments. FIG. 4C shows representative photographs showing MGMT^{P1401}-2A-GFP expression as detected by immuno-fluorescence using an anti-mouse MGMT specific antibody (in red) four weeks post-transduction of myoblasts to allow dilution of the IDLV vectors. Nuclei were stained with DAPI. The magnification shown is 200×. FIG. 4D is a schematic of the in vitro drug selection procedure using two populations of myoblasts with 3% or 35% of the CCR5 loci having undergone targeted integration, inserting the MGMT^{P140K}-2A-GFP gene into the endogenous CCR5 locus. FIG. 4E (left and right panels) shows the enrichment profile starting with respectively 3% or 35% of MGMT^{P140K}-2A-GFP positive cells following the indicated number of BG and BCNU drug selection cycles. Each value is a mean of three cultures.

FIG. 5, panels A through E, show ZFN mediated gene addition does not compromise the growth and fusion potential of human myoblasts in vitro and in vivo. FIG. 5A shows the proliferation of myoblasts expressed in terms of population doublings over time following their transduction with the indicated vectors. FIG. 5B shows Representative photographs of the fusion potential of myoblasts evaluated in vitro four weeks post-transduction. Myotube formation was determined by staining myoblasts for the expression of myosin heavy chain (MyHC) five days post-induction of differentiation using 2% serum containing medium. Nuclei were stained with DAPI. The magnification shown is 100×. FIG. 5C shows quantification of the fusion potential of myoblasts populations shown in panel B as determined by counting the proportion of nuclei forming MyHC-positive myotubes over the total number of nuclei from randomly selected fields. FIG. 5D shows Tibialis anterior muscles of immune-deficient NSG mice were transplanted with either non-transduced or microdystrophin-V5 gene-targeted myoblasts, and muscle fiber formation evaluated 4 weeks later. Human fibers were detected by immuno-fluorescence on successive cryosections using an anti-human dystrophin antibody (left-most panels) and gene-targeted fibers identified using an anti-V5 antibody (middle panels). Human nuclei were also stained with an anti-Lamin A/C antibody (right panels). The asterix show a fiber positive for both the full-length dystrophin and the micro-dystrophin gene. The arrows show a fiber positive only for the full-length dystrophin. FIG. 5E shows quantification of the number of fibers formed in vivo (as shown in FIG. 5D). Fibers were counted from muscle cryosections collected from n=6 transplanted muscles for each group. “NTC” refers to non-transduced cells.

### DETAILED DESCRIPTION

Described herein are methods and compositions useful for research and therapeutic applications for the development of reagents to introduce a gene into a cell for the treatment or prevention of a muscular dystrophy. These compositions and methods are useful for research and therapeutic applications and involve the use of genome editing via engineered nucleases to insert a therapeutic transgene at a safe harbor gene locus (e.g. AAVST, HPRT, CCR5 or Rosa). The dystrophin protein, expressed in muscle cells, plays a role in stabilizing a muscle fiber and connecting it to the cellular cytoskeleton. Lack of a functional dystrophin protein also leads to disassociation of the members of the DGC which acts as a link between the muscle fiber and the extracellular matrix, providing stability to the muscle cell. Thus, the introduced transgene for the treatment or prevention of a muscular dystrophy can be a dystrophin protein or a functional fragment and/or truncation thereof, or can be a gene encoding a member of the DGC if the cell comprises an endogenous DGC member gene that is mutated.

Adeno-associated virus-mediated in vivo gene transfer of the microdystrophin gene, a truncated but functional form of the protein, was recently shown to be feasible in dystrophic dogs (Wang et al, (2012) Mol Ther 20:1501-1507). Although promising, this strategy requires the in vivo administration of virus and immunosuppression of the host, procedures that can lead to multiple adverse effects. An alternative approach to in vivo gene transfer would be to use targeted integration to safely introduce the microdystrophin gene ex vivo into cultured myoblasts or muscle progenitor cells and then to transplant these cells into the patient (Skuk et al (2006) J Neuropathol Exp Neurol 65: 371-386 and Skuk et al (2004) Mol Ther 9:475-482). However, whether targeted gene transfer is feasible with high efficiency in human myoblasts is unknown. Here we demonstrate that ZFN-mediated targeted gene addition into human myoblasts of the enhanced green fluorescent protein (eGFP) or microdystrophin genes at the CCR5 locus can be done at high efficiency.

A major limitation of autologous myoblast transplantation for the treatment of DMD is that dystrophic myoblasts cannot be largely expanded ex vivo as they become rapidly senescent (Blau et al, (1983) Proc Natl Acad Sci USA 80:4856-4860). We believe this can be overcome by using induced pluripotent stem cells (iPSC) that can be efficiently differentiated into functional myogenic progenitors (Salani et al (2012) J Cell Mol Med 16:1353 Goudenege et al (2012) Mol Ther 20(11): 2153-2167). As such, a strategy where DMD patient fibroblasts would undergo (i) targeted integration of the dystrophin gene, (ii) differentiation into iPSC clones, (iii) whole genome sequencing and (iv) reprogramming into myogenic progenitors; could offer great promise for autologous cell therapy of DMD. Moreover, as this strategy would theoretically only require the isolation of a single clone with a validated targeted integration of the dystrophin gene, this would likely make it possible to achieve nuclease (e.g., ZFN)-driven targeted integration of the full length dystrophin gene. Indeed, the size of the dystrophin gene (14 kb) exceeds the encapsidation limit of IDLV vectors. However, in a scenario where the efficiency of homologous recombination would not be an issue, a large DNA fragment could be delivered to fibroblasts or myoblasts derived from DMD patients using standard transfection procedures as we have shown before (Quenneville et al (2004) Mol Ther 10:679-687). Moreover, we show that such efficiency can be easily increased by the in vitro selection of myoblasts following the co-addition of the mutated form of the O-6-methylguanine-DNA methyltransferase (MGMT^{P140K}) drug resistance gene. Finally, we also confirm that ZFN-modified myogenic cells preserve their potential to differentiate in vitro and in vivo upon intramuscular transplantation into immune-deficient mice.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526, incorporated by reference herein in its entirety.

Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Pat. No. 8,586,526.

A “selected” zinc finger protein or TALE is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197; WO 02/099084 and U.S. Pat. No. 8,586,526.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 20070218528, 2008/0131962 and 2011/0201055, incorporated herein by reference in their entireties.

The term “sequence” refers to a nucleotide sequence of any length, which can be DNA or RNA; can be linear, circular or branched and can be either single-stranded or double stranded. The teem “donor sequence” refers to a nucleotide sequence that is inserted into a genome. A donor sequence can be of any length, for example between 2 and 10,000 nucleotides in length (or any integer value there between or there above), preferably between about 100 and 1,000 nucleotides in length (or any integer there between), more preferably between about 200 and 500 nucleotides in length. “Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

A “chronic infectious disease” is a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeriamonocytogenes, and Mycobacterium tuberculosis.

The term “autoimmune disease” refers to any disease or disorder in which the subject mounts a destructive immune response against its own tissues. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes.

The term “cancer” as used herein is defined as a hyperproliferation of cells whose unique trait—loss of normal controls—results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the expression level of a gene. Modulation of expression can include, but is not limited to, gene activation and gene repression. Modulation may also be complete, i.e. wherein gene expression is totally inactivated or is activated to wildtype levels or beyond; or it may be partial, wherein gene expression is partially reduced, or partially activated to some fraction of wildtype levels. “Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a cleavage domain, the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site. Similarly, with respect to a fusion polypeptide in which a ZFP DNA-binding domain is fused to an activation or repression domain, the ZFP DNA-binding domain and the activation or repression domain are in operative linkage if, in the fusion polypeptide, the ZFP DNA-binding domain portion is able to bind its target site and/or its binding site, while the activation domain is able to upregulate gene expression or the repression domain is able to downregulate gene expression.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

The “dystophin associated complex” (DGC), also known as the “dystrophin associated protein complex” (DAPC) comprises several proteins, including dystrophin, the dystroglycans, the sarcoglycans, sacrospan, the α-dystrobrevins, the syntrophins, syncolin, nNOS, laminin-2, caveolin-3 and sodium channels (see Ehmsen et al, (2002) J Cell Sci 115:2801-2803). Several of these proteins, when aberrant, are also associated with autosomally inherited muscular dystrophies. For example, the sacroglycans, or SGs (αSG: limb girdle muscular dystrophy (LGMD) 2D, βSG: LGMD 2E, γSG: LGMD 2C, δSG: LGMD 2F), laminin-2: severe congenital muscular dystrophy MDC1A, and caveolin-3: LGMD-1C, hyperCKemia and rippling muscle disease. It will also be apparent that the term includes less than full length (but functional) proteins of the DGC, for example microdystrophin and/or minidystrophin.

DNA-Binding Domains

Described herein are compositions comprising a DNA-binding domain that specifically binds to a target site in a safe harbor gene. Any DNA-binding domain can be used in the compositions and methods disclosed herein, including but not limited to a zinc finger DNA-binding domain, a TALE DNA binding domain, the DNA-binding portion of a CRISPR/Cas nuclease, or a DNA-binding domain from a meganuclease.

In certain embodiments, the DNA binding domain comprises a zinc finger protein. Preferably, the zinc finger protein is non-naturally occurring in that it is engineered to bind to a target site of choice. See, for example, Beerli et al. (2002) Nature Biotechnol. 20: 135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties. In other embodiments, the DNA binding domain comprises a TALE DNA binding domain (see, U.S. Pat. No. 8,586,526, incorporated by reference in its entirety herein).

An engineered zinc finger or TALE DNA binding domain can have a novel binding specificity, compared to a naturally-occurring zinc finger or TALE protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In addition, as disclosed in these and other references, zinc finger domains and/or multi-fingered zinc finger proteins or TALEs may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned WO 02/077227.

Selection of target sites; ZFPs or TALEs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J Mol. Biol. 263:163-180; Argast et al. (1998) J Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117.128.

In certain embodiments, the DNA binding domain is an engineered zinc finger protein that binds (in a sequence-specific manner) to a target site in a safe harbor gene (e.g. CCR5, AAVS1, Rosa or albumin) and introduces a double strand break. Target sites typically include at least one zinc finger but can include a plurality of zinc fingers (e.g., 2, 3, 4, 5, 6 or more fingers). Usually, the ZFPs include at least three fingers. Certain of the ZFPs include four, five or six fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, wherein these regulatory domains can be transcriptional activation or repression domains.

In other embodiments, the DNA-binding domain comprises a naturally occurring or engineered (non-naturally occurring) TAL effector DNA binding domain See, e.g., U.S. Pat. No. 8,586,526, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al (2007) Science 318:648-651). These proteins contain a DNA binding domain and a transcriptional activation domain One of the most well characterized TALEs is AvrBs3 from Xanthomonas campestgri spv. Vesicatoria (see Bonas et al (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack et al. (2006) J Plant Physiol 163(3): 256-272). In addition, in the phytopathogenic bacteria Ralstoniasolanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

In certain embodiments, the nuclease comprises a CRISPR/Cas system. The CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems targeted to safe harbor and other genes are disclosed for example, in U.S. Provisional Application No. 61/823,689.

Thus, the nuclease comprises a DNA-binding domain in that specifically binds to a target site in any gene into which it is desired to insert a donor (transgene).

Target Sites

As described in detail above, DNA domains can be engineered to bind to any sequence of choice in a locus, for example a CCR5 gene, a Rosa gene, an albumin, an AAVS1 gene, an HRPT gene or other safe-harbor gene. See, e.g., U.S. Pat. Nos. 7,951,925 and 8,110,379; U.S. Publication Nos. 20080159996; 201000218264; 20120017290; 20110265198; 20130137104; 20130122591; 20130177983 and 20130177960 and U.S. Provisional Application No. 61/823,689. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Pat. No. 8,586,526.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237.

Selection of target sites; nucleases and methods for design and construction of fusion proteins (and polynucleotides encoding same) are known to those of skill in the art and described in detail in U.S. Patent Application Publication Nos. 20050064474 and 20060188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Pat. No. 8,586,526.

Donors

As noted above, insertion of an exogenous sequence (also called a “donor sequence” or “donor”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular (e.g., minicircle) form. See, e.g., U.S. Patent Publication Nos. 20100047805; 20110281361; and 20110207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor can be inserted so that its expression is driven by the endogenous promoter at the integration site, namely the promoter that drives expression of the endogenous gene into which the donor is inserted (e.g., highly expressed albumin, AAVS1, CCR5, HPRT etc. (see, U.S. Pat. Nos. 7,951,925 and 8,110,379; U.S. Publication Nos. 20080159996; 201000218264; 20120017290; 20110265198; 20130137104; 20130122591; 20130177983 and 20130177960). However, it will be apparent that the donor may comprise a promoter and/or enhancer, for example a constitutive promoter or an inducible or tissue specific promoter operably linked to the transgene.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus. See, e.g., U.S. patent publications 20080299580; 20080159996 and 201000218264.

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Fusion Proteins

Fusion proteins comprising DNA-binding proteins (e.g., ZFPs or TALEs) as described herein and a heterologous regulatory (functional) domain (or functional fragment thereof) are also provided. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, kinases, phosphatases, polymerases, endonucleases) and their associated factors and modifiers. U.S. Patent Application Publication Nos. 20050064474; 20060188987 and 2007/0218528 for details regarding fusions of DNA-binding domains and nuclease cleavage domains, incorporated by reference in their entireties herein.

### Nucleases

In certain embodiments, the fusion protein comprises a DNA-binding binding domain and cleavage (nuclease) domain, for example ZFN, TALEN and/or CRISPR/Cas system. As such, gene modification can be achieved using a nuclease, for example an engineered nuclease. Engineered nuclease technology is based on the engineering of naturally occurring DNA-binding proteins. The methods and compositions described herein are broadly applicable and may involve any nuclease of interest. Non-limiting examples of nucleases include meganucleases, TALENs, zinc finger nucleases and CRISPR/Cas nucleases. In certain embodiment, the nuclease is a meganuclease (homing endonuclease). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-SceIV, I-CsmI, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

DNA-binding domains from naturally-occurring meganucleases, primarily from the LAGLIDADG family, have been used to promote site-specific genome modification in plants, yeast, Drosophila, mammalian cells and mice, but this approach has been limited to the modification of either homologous genes that conserve the meganuclease recognition sequence (Monet et al. (1999), Biochem. Biophysics. Res. Common. 255: 88-93) or to pre-engineered genomes into which a recognition sequence has been introduced (Route et al. (1994), Mol. Cell. Biol. 14: 8096-106; Chilton et al. (2003), Plant Physiology. 133: 956-65; Puchta et al. (1996), Proc. Natl. Acad. Sci. USA 93: 5055-60; Rong et al. (2002), Genes Dev. 16: 1568-81; Gouble et al. (2006), J. Gene Med. 8(5):616-622). Accordingly, attempts have been made to engineer meganucleases to exhibit novel binding specificity at medically or biotechnologically relevant sites (Porteus et al. (2005), Nat. Biotechnol. 23: 967-73; Sussman et al. (2004), J Mol. Biol. 342: 31-41; Epinat et al. (2003), Nucleic Acids Res. 31: 2952-62; Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication Nos. 20070117128; 20060206949; 20060153826; 20060078552; and 20040002092). In addition, naturally-occurring or engineered DNA-binding domains from meganucleases have also been operably linked with a cleavage domain from a heterologous nuclease (e.g., FokI).

In other embodiments, the nuclease is a zinc finger nuclease (ZFN). ZFNs comprise a zinc finger protein that has been engineered to bind to a target site in a gene of choice and cleavage domain or a cleavage half-domain.

As noted above, zinc finger binding domains can be engineered to bind to a sequence of choice. See, for example, Beerli et al. (2002) Nature BiotechnoL 20:135-141; Pabo et al. (2001) Ann. Rev. Biochem. 70:313-340; Isalan et al. (2001) Nature BiotechnoL 19:656-660; Segal et al. (2001) Curr. Opin. Biotechnol 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

Selection of target sites; ZFNs and methods for design and construction of fusion proteins (and polynucleotides encoding same) are known to those of skill in the art and described in detail in U.S. Patent Application Publication Nos. 20050064474 and 20060188987, incorporated by reference in their entireties herein.

In addition, as disclosed in these and other references, zinc finger domains and/or multi-fingered zinc finger proteins may be linked together using any suitable linker sequences, including for example, linkers of 5 or more amino acids in length. See, e.g., U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In some embodiments, the nuclease is an engineered TALEN. Methods and compositions for engineering these proteins for robust, site specific interaction with the target sequence of the user's choosing have been published (see U.S. Pat. No. 8,586,526).

In other embodiments, the nuclease is a CRISPR/Cas nuclease, as described above.

Nucleases such as ZFNs, TALENs and/or meganucleases also comprise a nuclease (cleavage domain, cleavage half-domain) As noted above, the cleavage domain may be heterologous to the DNA-binding domain, for example a zinc finger or TALE DNA-binding domain and a cleavage domain from a nuclease or a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type ITS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-FokI or TALE-FokI fusions, two fusion proteins, each comprising a FokI cleavage half-domain, can be used to reconstitute a catalytically active cleavage domain Alternatively, a single polypeptide molecule containing a zinc finger or TALE DNA binding domain and two FokI cleavage half-domains can also be used. Parameters for targeted cleavage and targeted sequence alteration using zinc finger- or TALE-FokI fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987 and 20080131962, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

Exemplary engineered cleavage half-domains of FokI that form obligate heterodimers include a pair in which a first cleavage half-domain includes mutations at amino acid residues at positions 490 and 538 of FokI and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., Example 1 of co-owned U.S. Patent publication No. 20080131962 (filed May 23, 2007), and issued U.S. Pat. No. 7,914,796, the disclosures of which are incorporated by reference in their entirety for all purposes.

In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type Fold), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type Fold), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). (See US Patent Publication No. 20110201055). Engineered cleavage half-domains described herein can be prepared using any suitable method, for example, by site-directed mutagenesis of wild-type cleavage half-domains (FokI) as described in U.S. Patent Publication Nos. 20050064474; 20080131962; and 20110201055.

Alternatively, nucleases may be assembled in vivo at the nucleic acid target site using so-called “split-enzyme” technology (see, e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Engineered cleavage half-domains described herein can be prepared using any suitable method, for example, by site-directed mutagenesis of wild-type cleavage half-domains (FokI) as described in Example 5 of U.S. Patent Publication No. 20050064474; 20070305346; 20080131962; 20110201055; and 20120142062.

Nuclease expression constructs can be readily designed using methods known in the art. See, e.g., United States Patent Publications 20030232410; 20050208489; 20050026157; 20050064474; 20060188987; 20060063231; and International Publication WO 07/014275. In certain embodiments, expression of the nuclease is under the control of an inducible promoter, for example the galactokinase promoter which is activated (de-repressed) in the presence of raffinose and/or galactose and repressed in presence of glucose. In particular, the galactokinase promoter is induced and the nuclease(s) expressed upon successive changes in the carbon source (e.g., from glucose to raffinose to galactose). Other non-limiting examples of inducible promoters include CUP1, MET15, PHO5, and tet-responsive promoters.

Delivery

The nucleases (e.g., ZFPs, TALEs, CRISPR/Cas) and/or donors, may be delivered to a target cell by any suitable means in protein and/or polynucleotide form.

Methods of delivering proteins comprising zinc finger proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases as described herein may also be delivered using vectors containing sequences encoding one or more of the DNA-binding proteins or sequences. Donor encoding polynucleotides may be similarly delivered. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more nucleases (or the same or different types) and/or one or more donors. Thus, when one or more ZFPs, TALEs and/or CRISPR/Cas nucleases and one or more donors are introduced into the cell, the nucleases and/or donors may be carried on the same vector or on different vectors. When multiple vectors are used, each vector may comprise a sequence encoding one or multiple nucleases and/or donors.

Conventional viral and non-viral based gene transfer methods can be used to introduce nucleic acids encoding nucleases and/or donors in cells (e.g., mammalian cells) and target tissues. Such methods can also be used to administer nucleic acids encoding nucleases and/or donors to cells in vitro. In certain embodiments, nucleic acids encoding nucleases and/or donors are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaud British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et at (2009) Nature Biotechnology 27(7) p. 643).

The use of RNA or DNA viral based systems for the delivery of nucleic acids encoding engineered nucleases and/or donors take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polynucleotides include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type virus. The vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6 and AAV8, AAV 8.2, AAV9, and AAV rh10 and pseudotyped AAV such as AAV2/8, AAV2/5 and AAV2/6 can also be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, AAV, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see US Pat. No. 7,479,554.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a ZFP nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells (iPSCs), hematopoietic stem cells, neuronal stem cells and mesenchymal stem cells. In certain embodiments, the cells are myoblasts. The myoblasts may be derived from stem cells, for example, iPSCs including from iPSCs derived from patients with muscular disorders such as muscular dystrophy. See, e.g., U.S. Patent Publication No. 20120252122 regarding production of nuclease-modified iPSCs from patient derived cells.

In one embodiment, stem cells, for example iPSCs derived from are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and Tad (differentiated antigen presenting cells) (see, Inaba et al., J. Exp. Med. 176:1693-1702 (1992)). Stem cells may be also derived from muscles. Skeletal muscle is a convenient source for somatic stem cells and contains several distinct populations of myogenic stem cells including satellite cells that are mainly responsible for muscle growth and regeneration, and multipotent muscle-derived stem cells (MDSCs). MDSCs display the characteristics of long-term proliferation, high self-renewal and a superior capacity to regenerate skeletal muscle (Usas et al (2011) Medicina (Kaunas) 47(9): 469-79).

Stem cells that have been modified may also be used in some embodiments. For example, stem cells that have been made resistant to apoptosis may be used as therapeutic compositions where the stem cells also contain the nucleases and/or donors of the invention. Resistance to apoptosis may come about, for example, by knocking out BAX and/or BAK using BAX- or BAK-specific nucleases (see, U.S. Patent Publication No. 20100003756) in the stem cells, or those that are disrupted in a caspase, again using caspase-6 specific nucleases for example.

Vectors (e.g., retroviruses, adenoviruses, liposomes, etc.) containing nucleases and/or donor nucleic acids can also be administered directly to an organism for transduction of cells in vivo. Alternatively, naked DNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34⁺ cells, include adenovirus Type 35.

Vectors suitable for introduction of transgenes into immune cells (e.g., T-cells) include non-integrating lentivirus vectors (IDLVs). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

Applications

The disclosed compositions and methods can be used for any application in which it is desired to express a protein related to a muscular dystrophy. In particular, these methods and compositions can be used where it is desired to express a dystrophin protein, including but not limited to, therapeutic and research applications. The invention also contemplates the insertion of DNA sequences encoding a protein related to a muscular dystrophy in a stem cell. The methods and compositions of the invention can be used to treat various muscular dystrophy diseases, including but not limited to, Duchenne's muscular dystrophy, Becker's muscular dystrophy, and limb girdle muscular dystrophy. Treatment of these diseases can be accomplished by introduction of modified stem cells (e.g., iPSCs such as patient derived iPSCs) into a subject and/or through introduction of semi-differentiated myoblasts.

Cells may be introduced in specific locations such as into major skeletal muscles, or into smaller discrete muscles such as those involved in finger and/or thumb control, or into the essential diaphragm and intercostals muscles that are critical for patient survival (Usas, ibid). DMD arises from numerous distinct mutations located in various exons of the dystrophin gene. The design of nucleases specific for the dystrophin gene could theoretically allow for the genetic correction of each individual mutation, keeping the expression of the dystrophin under its endogenous promoter. However, this strategy would require the development of several nucleases to account for the diversity of mutations found in the patient population. On the other hand, targeted gene addition at a safe-harbor locus (e.g., CCR5) could offer a unique solution to DMD patients.

Here, we found that nuclease-mediated targeted integration of the microdystrophin gene can be performed ex vivo in human myoblasts at a sufficiently high frequency to be considered a valid approach for the treatment of myogenic diseases. Indeed, using a CCR5-specific ZFN pair, our results showed that over 40% targeted gene addition can be obtained in human myoblasts in the absence of any apparent cytotoxicity. A major property of myoblasts is their capacity to fuse with each other or with existing fibers to regenerate damaged muscles. As such, our results showed that the fusion potential of nuclease-targeted myoblasts is not altered in vitro or in vivo following transplantation in mice.

An important observation from our work is that the constitutive expression of the microdystrophin gene under the control of the ubiquitous PGK promoter was not toxic to human myoblasts, making the use of a muscle specific promoter unnecessary for the in vitro expansion of modified myoblasts. This is likely explained by the fact that in nuclease-modified cells limited expression of the microdystrophin gene is observed from a single site-specific targeted locus. In contrast, when lentiviral vectors are used to randomly insert the microdystrophin gene, microdystrophin expression is likely higher and toxic to mononuclear cells due to insertion of multiple copies of the gene (Quenneville et al (2007) Mol Ther 15:421-438).

The methods and compositions of the invention are also useful for the design and implementation of in vitro and in vivo models, for example, animal models of muscular dystrophies, which allows for the study of these disorders and furthers discovery of useful therapeutics.

The following Examples relate to exemplary embodiments of the present disclosure in which the nuclease comprises a ZFN. It will be appreciated that this is for purposes of exemplification only and that other nucleases can be used, for instance TALENs, CRISPR/Cas nucleases, homing endonucleases (meganucleases) with engineered DNA-binding domains and/or fusions of naturally occurring or engineered homing endonucleases (meganucleases) DNA-binding domains and heterologous cleavage domains

### EXAMPLES

### Example 1

### Targeted Addition of the eGFP or Microdystrophin Gene in Human Myoblasts

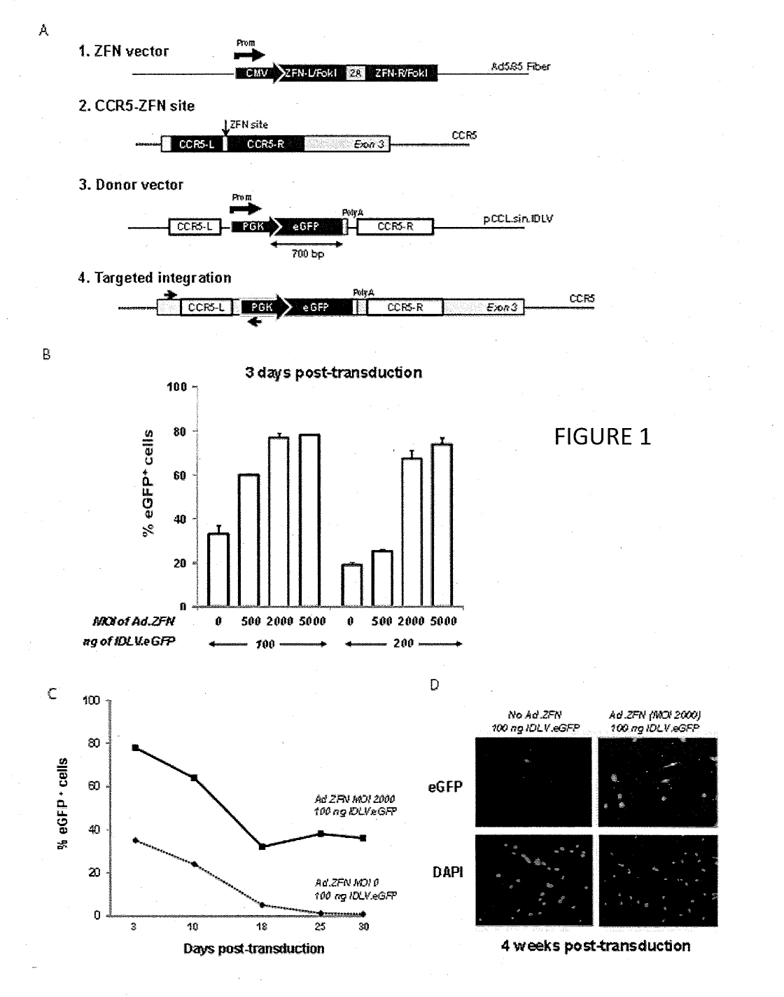

We selected the CCR5 locus to carryout ZFN-mediated targeted gene addition in human myoblasts. This is justified by the fact that highly specific ZFN targeting this locus (see U.S. Pat. No. 7,951,925) have already been developed and are currently being used in the clinic to disrupt the CCR5 locus in autologous CD4 T cells from HIV patients (clinicaltrials.gov identifier NCT00842634). We have also previously shown that we can achieve adequate transgene expression from genes inserted into the endogenous CCR5 locus using the CCR5 ZFN (Lombardo et al, (2007) Nat Biotech 25(11):1298-1306, Lombardo et al (2011) Nat Methods. 8(10):861-9). These two characteristics greatly increase the translational potential of our approach. Using a non-integrating chimeric adenoviral vector (Ad5/F35) for the transient expression of CCR5 ZFNs and integrase defective lentiviral vector (IDLV), carrying a PGK-eGFP template DNA flanked by CCR5 homology arms (FIG. 1A), we first determined the best vector ratio necessary to achieve targeted-gene addition in human myoblasts (FIG. 1B). We observed a high-frequency (close to 40%) of targeted-gene addition, in the absence of any drug selection, when myoblasts were simultaneously exposed to the Ad5/F35 ZFN vector at an MOI of 2000 and to 100 ng of p24 IDLV.GFP donor (FIG. 1C and D). Targeted gene addition was determined by flow cytometry at selected intervals up to 30 days post-transduction, the minimum time required to dilute out the residual eGFP expression from non-integrated IDLV.

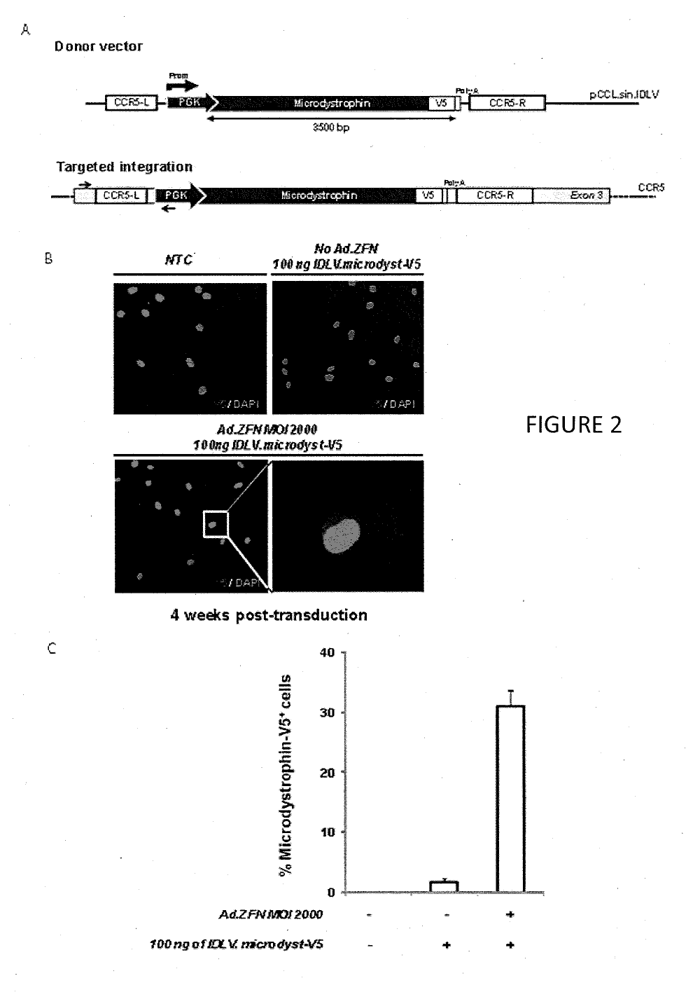

We next wanted to determine if it was possible to introduce, with a similar efficiency, a DNA template containing the microdystrophin gene that is about five times the size of the eGFP gene (FIG. 2A). Using the same optimized transduction protocol described above, we achieved up to 30% targeted addition of the microdystrophin gene into human myoblasts, a level obtained without any drug selection. Targeted integration was determined by immuno-cytochemistry four weeks post-transduction (FIG. 2B and C).

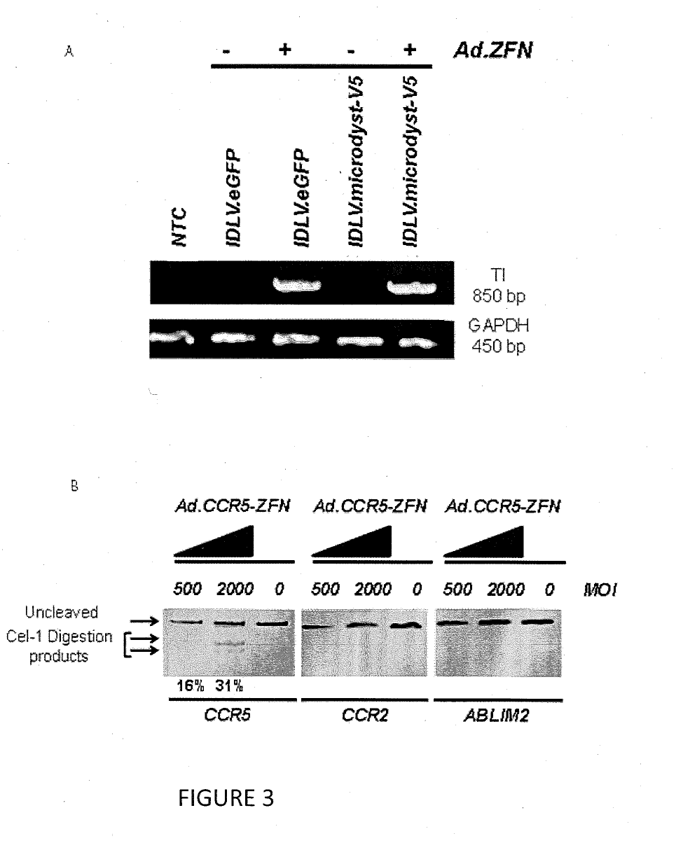

Importantly, we also confirmed that targeted integration of both the eGFP and microdystrophin genes occurred at the CCR5 locus only in cells that were co-transduced with vectors delivering both ZFNs and the template DNA. This was established by PCR using specific primers recognizing the 5′ integration junction (FIG. 3A).

The specificity of the CCR5-ZFN was also determined by performing the surveyor nuclease Cel-1 assay at the CCR2 and ABLIM2 loci, the two most frequent off-target loci identified for this ZFN pair. As expected, and in opposition to the CCR5 locus where we could detect over 30% cleavage activity, no cleavage was observed at these off-target loci, confirming the specificity of the CCR5-ZFN in myoblasts (FIG. 3B).

### Example 2

### Targeted Integration of the MGMT^{P140K} Drug Resistance Gene Allows for the In Vitro Selective Enrichment of Modified Myoblasts

To maximize the chance of success of cell therapy, it certainly would be advantageous to obtain a population of myoblasts that would be nearly if not fully gene-modified. Hence, despite the remarkably high level of targeted gene addition we achieved in myoblasts in absence of drug selection, we explored the possibility of co-targeting the 06-methylguanine-DNA methyltransferase^{P140K} (MGMT^{P140K}) gene, which expression can confer a selective growth advantage both in vitro and in vivo (Lee et al (2009) Stem Cells 27:1098). In brief, cells which express the MGMT^{p140K} gene, have a selective growth advantage when exposed to the wild-type MGMT inhibitor 06-benzylguanine (BG) in combination with a low dose of 1,3-bis(2-chloroethyl)-N-nitrosourea (BCNU).

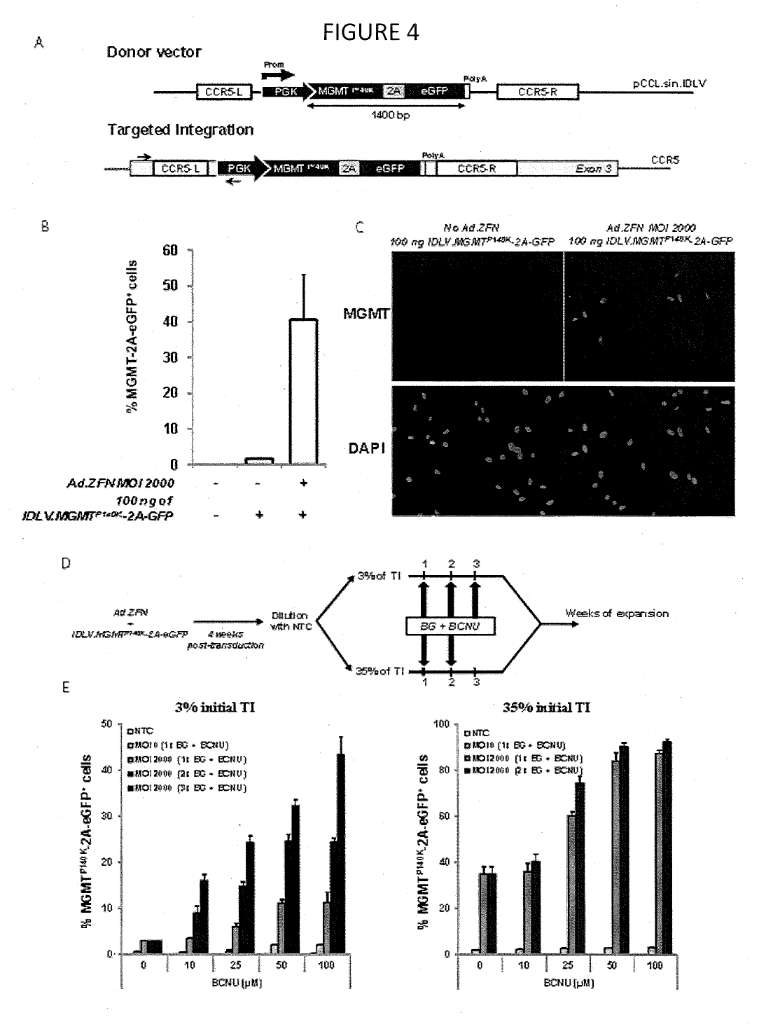

Consequently, we next verified whether expression from the integrated MGMT^{P140K} gene, along with the eGFP reporter gene to facilitate tracking of transduced cells, was sufficient to confer myoblasts protection against treatments with BG and BCNU (FIG. 4A). Using IDLV to deliver a MGMT^{P140K}-eGFP template DNA flanked by CCR5 homology arms, we obtained 40-50% gene addition in myoblasts in the absence of drug selection (FIG. 4B and C). However, to fully evaluate the selection potential of the BG/BCNU combination, we thought it would be more appropriate to work with a population containing a much lower level of gene addition. As such, we generated two populations of gene-targeted myoblasts containing either 3% or 35% of cells expressing the MGMT^{P140K}-2A-GFP gene using non-transduced cells for dilution. Both populations were exposed to 20 μM BG for one hour followed by increasing doses of BCNU (10, 25, 50 and 100 μM) for two hours (FIG. 4D).

Myoblasts were then allowed to recover for one week after which the proportion of eGFP positive cells was analyzed by flow cytometry, a procedure defined as one cycle of drug selection. Our results demonstrate that the proportion of MGMT^{P140K}-2A-GFP expressing cells increased from 3 to 11% following only one cycle using 100 μM BCNU (FIG. 4D). After three cycles of selection using the same concentration of BCNU, up to 45% of the myoblasts were positive for eGFP, representing a 15-fold enrichment over the initial population. However, additional cycle of selection did not allow us to further increase the proportion of eGFP positive cells, suggesting acquired resistance of non-modified cells against BCNU toxicity. In comparison, when starting with a population containing already 35% of gene-targeted myoblasts, over 85% of the cells were expressing eGFP after only one cycle of selection using 50 μM of BCNU (FIG. 4D).

Altogether these results suggest that the efficiency of ZFN-mediated gene addition at the CCR5 locus, when combined with one round of in vitro drug selection, is similar to the one obtained using a VSVG-pseudotyped lentiviral integrating vector.

### Example 3

### ZFN-Driven Gene Addition Mediates No Cytotoxicity in Human Myoblasts

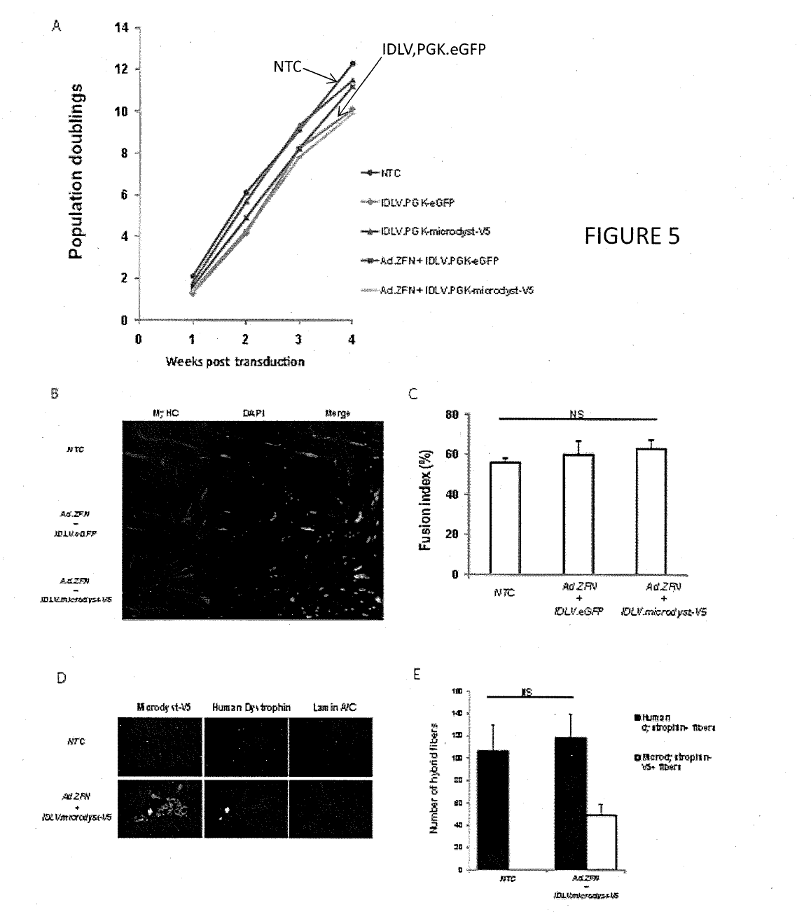

We next wanted to verify the impact that the co-transduction (Ad5/35 and IDLV) and subsequent transient expression of ZFNs may have on the growth and function of myoblasts. We first found that when passaged every three days for up to four weeks post-transduction, that gene-targeted myoblasts experience very little cytotoxicity based on the kinetics of their population doublings (FIG. 5A). Modified myoblasts were also placed for five days in medium containing 2% serum and their ability to fuse in vitro evaluated by immuno-cytochemistry based on the expression of the muscle heavy chain myosin (MyHC) (FIG. 5B). As for cell division, we found ZFN-mediated gene addition did not interfere with MyHC expression or fusion potential of myoblasts in vitro (FIG. 5C).

### Example 4

### Engraftment of Gene Targeted Human Myoblasts into the Muscle of Immune-Deficient Mice

To further assess the potential for gene-targeted myoblasts to serve as a potential cell therapy, we also measured their capacity to form muscle fibers in vivo. We found that ZFN-modified myoblasts that express the microdystrophin gene were as competent as their non-modified counterparts in forming muscle fibers when transplanted in the Tibialis anterior muscle of immune-deficient NSG mice (FIG. 5D and E). Myoblasts engraftment was determined by immuno-histochemistry performed on cryosections of muscle stained with an antibody against the V5 epitope, which was fused at the 3′ end of the microdystrophin gene (see Pichavant et al, (2010) Mol Ther 18: 1002-1009) (FIG. 2A). Indeed, we found the ratio of V5 positive fibers over the total number of fibers formed (as detected by staining for the full length human dystrophin) to be similar to the level of targeted gene addition observed prior to the transplantation (FIG. 5E).

Altogether, these results suggest that ZFN-mediated gene addition does not interfere with the functionality of myoblasts in vitro and in vivo.

### Example 5

### Materials and Methods

### Mice

NOD/LtSz-scid/IL2r^{γ+/+} (NSG) mice were obtained from the Jackson Laboratory and housed in the animal care facility at the CHU Sainte-Justine Research Centre under pathogen-free conditions in sterile ventilated racks. All in vivo manipulations were previously approved by the institutional committee for good laboratory practices for animal research (GLPAR) (protocol number S10-32).

### Vectors and Lentivirus Preparation

ZFNs targeting exon 3 of the human CCR5 gene were previously described (see co-owned U.S. Pat. No. 7,951,925). A non-replicating, chimeric adenovirus encoding the CCR5-ZFNs was generated using the human adenovirus 5 vector with the fiber shaft and knob domains replaced with the domains from human species B adenovirus 35 (Ad5/F35) (see Perez et al, (2008) Nat Biotechnol 26:808-816). Integrase defective lentiviral vectors (IDLV) carrying GFP, microdystrophin-V5 (as described above) or MGMT^{P140K}-2A-GFP donor cassettes were generated from the HIV-derived self-inactivating third-generation transfer construct pCCLsin.cPPT.hPGK.X.BGHpA using an integrase-defective packaging plasmid. IDLV stocks were prepared as described elsewhere. Lentiviral particles were quantified upon concentration by ultra-centrifugation by HIV-1 Gag p24 Antigen ELISA (ZeptoMetrix Corporation). Yields ranged from 5-20 ng p24/μl, depending on the vector type.

### Human Myoblast Culture and Transduction

Human myoblasts were obtained from a postmortem muscle biopsy of a normal 13-month-old male and proliferated in MB-1 medium (Hyclone—X) supplemented with 15% fetal bovine serum (Wisent—Saint-Bruno, QC, Canada), 1% penicillin-streptomycin (Wisent), 10 μg/l of bFGF (R and D systems—Burlington, ON, Canada), 0.4 mg/l of dexamethasone (Sigma —Oakville, ON, Canada), and 5 mg/l of insulin (Sigma). Myoblasts were exposed simultaneously to Ad5/F35-ZFN (MOI range of 500-2000) and IDLV template DNA vectors (100 or 200 ng of p24 per 10⁵ cells) for 18 hours, a procedure that was repeated twice. Transduced cells were then expanded in growth conditions and passaged every 4 days until analysis. For differentiation, cells were expanded in DMEM medium supplemented with 2% FBS and antibiotics for 5 days, myotubes were first fixed in 4% paraformaldehyde for 15 min, permeabilized 3 times for 15 min with 3% triton X-100 in PBS and then immuno-stained against the mouse anti-myosin heavy chain (MyHC) using the MF20 anti-mouse MyHC antibody at a dilution of (1:100) for 2 h (Developmental Studies Hybridoma Bank, University of Iowa) and subsequently with an anti-mouse ALEXA fluor 594 at a dilution of (1:300) for 1 h (Invitrogen—Burlington, ON, Canada). Nuclei were counterstained with DAPI. The fusion index (defined as the number of DAPI stained nuclei inside MyHC⁺ myotubes in a given field divided by the total number of DAPI stained nuclei in the same field) of each condition was calculated. The assay was done 3 times.

### Cel-1 Assay

The ability of the CCR5-ZFN pair to cut the CCR5 locus was verified using the surveyor nuclease (Cel-1 from Transgenomic—Omaha, Nebr., USA), a nucleotide mismatch selective endonuclease able to detect the presence of mutant alleles. Briefly the mismatch assay consists of amplifying the target region from ZFN-treated genomic DNA via PCR using the following specific primers (CCR5 Cel-1 Forw primer: 5′-AAGATGGATTATCAAGTGTCAAGTCC-3′ (SEQ ID NO:1); CCR5 Cel-1 Rev primer: 5′-CAAAGTCCCACTGGGCG-3′ (SEQ ID NO:2); CCR2 Cel-1 Forw primer: 5′-CCACATCTCGTTCTCGGTTTATC-3′ (SEQ ID NO:3); CCR2 Cel-1 Rev primer: 5′-CGCCAAAATAACCGATGTG-3′ (SEQ ID NO:4) and ABLIM2 Cel-1 Forw primer: 5′-CGATGACTCTGAGGTCTACTCG-3′ (SEQ ID NO:5); ABLIM2 Cel-1 Rev primer: 5′-CAAGTGAACACATGGTTTGCAG-3′ (SEQ ID NO:6)) as described previously. PCR products are denatured and allowed to re-anneal. The mismatch sensitive enzyme cuts DNA at the sites where heterogeneous mismatches occur. Reactions are resolved by gel electrophoresis. The presence of digested PCR products indicates mutagenesis due to ZEN-induced cleavage. The assay is sensitive enough to detect single-nucleotide changes and has a linear detection range between 0.69 and 44%.

### Analysis of Targeted Gene Addition Efficacy

Efficacy of eGFP gene-targeted integration was determined at day 3, 10, 18, 25 and 30 days post-transduction by immuno-fluorescence using an Olympus BX51 epifluorescent microscope and by flow cytometry (LSR Fortessa cell analyzer, BD). Non-viable cells were identified using 7-AminoActinomicin D (7-AAD) and were excluded from the analysis. Microdystrophin-V5 expression was detected by immunofluorescence using an anti-V5 antibody (1:5000; Invitrogen) 1 month after transduction. Targeted integration at the CCR5 locus was determined by PCR using 100 ng of genomic DNA with a set of primers against the 5′ junction (Forw CCR5 primer: 5′-TTGGAGGGGTGAGGTGAGAGG-3′ (SEQ ID NO:7), Rev hPGK primer: 5′-TGAAGAATGTGCGAGACCCAGG-3′ (SEQ ID NO:8)) as follows: 94° C. for 10 min, then 30 cycles of 94° C. for 1 min, 60° C. for 30 sec and 72° C. for 1 min, followed by extension at 72° C. for 10 min. The expected amplicon length is 815 bp.

### In Vitro Selection of Myoblasts Using the BCNU/BG Combination

Four weeks post-transduction, gene modified myoblasts containing the MGMT^{P140K}-2A-GFP cassette inserted at the endogenous CCR5 locus were treated with 50 μM O₆BG for two hours followed by increasing doses (range from 10-100 μM) of BCNU for one hour. Cells were then washed with PBS and allowed to proliferate in the presence of 50 O₆BG for seven days before being analyzed by FACS. Where indicated, this procedure was repeated up to two more times. Cells were maintained in the presence of O₆BG during their post-treatment expansion to inactivate the endogenous MGMT gene.

### In Vivo Transplantation of Human Myoblasts

12 week-old NSG immune-deficient mice were irradiated at a dose of 9 Gy (1 Gy per minutes) using a Faxitron model CP160 at the leg level to inhibit the proliferation of the recipient satellite cells, a condition that favors the participation of grafted cells in recipient muscle regeneration. The day of the transplantation, cultured myoblasts were detached from the flasks using 0.25% Trypsin-EDTA solution and washed twice with PBS. A total of 1×10⁶ myoblasts resuspended in 20 μl of PBS containing 10 μg/ml cardiotoxin (Sigma), were implanted in each Tibialis anterior (TA) through about 20 percutaneous microinjections. The grafted TA muscles were harvested 4 weeks after transplantation, embedded in OCT and snap frozen in liquid nitrogen.

### Immune-Detection of Hybrid Fibers in Muscle Sections

Muscle frozen sections (12 μm) were first washed with PBS and non-specific binding was blocked by incubating the sections with PBS containing 10% FBS for 1 h. Immuno-fluorescence to detect human nuclei was performed with a mouse anti-human Lamin A/C antibody (1:100 for 2 h; Vector Laboratories—Burlington, ON, Canada). Incubation with the primary antibody was followed by incubation with an anti-mouse ALEXA fluor 549 (1:300 for 1 h; Invitrogen). Myofibers expressing the microdystrophin-V5 protein were detected using a mouse anti-V5 antibody (1:200 overnight; Invitrogen) and an anti-mouse ALEXA fluor 488 (1:300 for 1 h; Invitrogen). All sections were mounted using the Vectashield (Vector Laboratories) mounting medium to prevent loss of fluorescence.

### Statistical Analysis

All data are expressed as means±SEM and are representative of at least three separate experiments. The statistical significance of the difference between groups was determined by a Student's t-test using GraphPad software. A value of P<0.05 was considered significant.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A myoblast comprising a transgene encoding a dystophin-associated glycoprotein complex (DGC) protein, wherein the transgene is integrated site-specifically using a nuclease into an endogenous safe harbor gene.

2. The myoblast of claim 1, wherein the myoblast is derived from an induced pluripotent stem cell (iPSC).

3. The myoblast of claim 1, wherein the endogenous safe harbor gene is selected from the group consisting of CCR5, HPRT, AAVS1, Rosa and albumin.

4. The myoblast of claim 1, wherein the transgene encodes a dystrophin protein or functional fragment thereof.

5. The myoblast of claim 4, wherein the dystrophin protein is a minidystrophin or microdystrophin protein.

6. A method of increasing expression of a dystrophin gene in a subject, the method comprising administering a myoblast according to claim 1 to the subject.

7. The method of claim 6, wherein the subject has a muscular dystrophy.

8. The method of claim 6, wherein the myoblast is derived from an induced pluripotent stem cell (iPSC).

9. The method of claim 6, wherein the endogenous safe-harbor gene is selected from the group consisting of CCR5, HPRT, AAVS1, Rosa and albumin.

10. The method of claim 6, wherein the transgene encodes a dystrophin protein.

11. The method of claim 10, wherein the dystrophin protein is a minidystrophin or microdystrophin protein.
